(19) United States
(12) Patent Application Publication
(10) Pub. No.: US 2024/0299122 A1
(43) Pub. Date: Sep. 12, 2024

(54) SURGICAL IMAGING SYSTEM WITH SELECTIVE VIEW

(71) Applicant: Arthrex, Inc., Naples, FL (US)

(72) Inventors: Michael Dominik Steiner, Canton, MA (US); David Selby, Santa Barbara, CA (US); Efrain O. Morales Correa, Santa Ynez, CA (US); Kevin Vallance, Ventura, CA (US); Craig Speier, Santa Barbara, CA (US); John Hygelund, Santa Barbara, CA (US)

(73) Assignee: Arthrex, Inc, Naples, FL (US)

(21) Appl. No.: 18/598,665

(22) Filed: Mar. 7, 2024

Related U.S. Application Data
(60) Provisional application No. 63/450,509, filed on Mar. 7, 2023.

(51) Int. Cl. A61B 90/00

(52) U.S. Cl. CPC A61B 90/361; A61B 90/37; A61B 2090/3612; A61B 2090/364; A61B 2090/373

(57) ABSTRACT

A surgical imaging system includes a scope having an optic element aligned at a first inclination angle defining a native perspective. An image sensor is configured to capture source image data in a field of view transmitted through the optic element. A controller is configured to control the capture of source image data at the native perspective at the first inclination angle. The controller further selects a first subset of the source image including a first portion of the field of view simulating a second inclination angle and selectively outputs modified source image data and the first image data to a display screen.

### CROSS-REFERENCE TO RELATED APPLICATIONS

This application claims priority under 35 U.S.C. § 119 (e) and the benefit of U.S. Provisional Application No. 63/450,509 entitled SURGICAL IMAGING SYSTEM WITH SELECTIVE VIEW, filed on Mar. 7, 2023, by Steiner et al., the entire disclosure of which is incorporated herein by reference.

### BACKGROUND

The present disclosure generally relates to a surgical imaging system and, more particularly, to a surgical camera and control system configured to selectively adjust a perspective of a field of view. Modern day surgical procedures apply a variety of advanced techniques to improve patient outcomes. However, such procedures may increase the complexity of the procedures and correspondingly increase the number of specialized tools and related equipment necessary to complete procedures. The disclosure provides for an improved imaging system that may improve the operation associated with some of these procedures.

### SUMMARY

Surgical imaging systems have evolved to include a variety of specialty devices, including cameras and imaging devices designed to access remote patient cavities. Such devices may include specialty optics and lenses that may be provided with different inclination angles, allowing the devices to capture fields of view that may target anatomical features at the corresponding inclination angles. While interchangeable scopes with different inclination angles may provide for highly specialized operation, they may also require removal and exchange during a surgical procedure to provide such functionality. In various implementations, the disclosure provides for a surgical imaging system comprising a scope having an optical element aligned at an inclination angle defining a native perspective. As discussed in detail in the following examples, a controller of the imaging system may process the source image data captured at the native perspective of the scope and selectively generate modified image data having a simulated perspective angle relative to the inclination angle and the native perspective.

In operation, the scope of the surgical imaging system may capture the source image data at the inclination angle throughout the various modes of operation, such that a resolution or dimensions of the field of view are consistently captured and processed to provide the operation discussed herein. For example, in various embodiments, the scope may include an optic element aligned at an inclination angle at a distal end oriented at approximately 45°. This orientation may define the native perspective associated with the scope and the optic element relative to a scope axis along which the source image data is captured. Based on the source image data captured at the native perspective, the controller of the system may output the display data having the original perspective or selectively generate modified image data having a simulated perspective angle offset and different from the native perspective. As discussed in various detailed examples in the following description, the modified source image data having the simulated perspective angle may correspond to modified image data that may be generated by remapping light rays aligned with pixels within the field of view to simulate image data having a different angle of incidence and offset center ray aligned with the simulated perspective angle. Accordingly, the disclosed systems and methods may not only adjust or crop a portion of the field of view for display, the systems may further provide for the selective modification of the source image data to appear to have been captured from a different perspective offset from the native perspective.

These and other features, objects and advantages of the present disclosure will become apparent upon reading the following description thereof together with reference to the accompanying drawings.

### BRIEF DESCRIPTION OF THE DRAWINGS

FIG. 1 is an illustrative diagram demonstrating a surgical imaging system;

FIG. 2A is a diagram demonstrating simulated image data captured from a native perspective;

FIG. 2B is a diagram demonstrating image data modified to have a simulated perspective that is angled relative to the native perspective shown in FIG. 2A;

FIG. 2C is a diagram demonstrating image data modified to have a simulated perspective that is angled relative to the native perspective shown in FIG. 2A;

FIG. 2D is flow chart demonstrating a method for generating image data at a simulated perspective;

FIG. 3A is a schematic diagram representing the incidence of a plurality of rays of light on an optic element at a native perspective;

FIG. 3B is a schematic diagram demonstrating a plurality of modified rays of light generated to simulated modified image data having a simulated perspective offset relative to the native perspective demonstrated in FIG. 3A;

FIG. 3C is a schematic diagram demonstrating a plurality of modified rays of light generated to simulated modified image data having a simulated perspective offset relative to the native perspective demonstrated in FIG. 3A;

FIG. 4 is a representative diagram demonstrating modified image data having a simulated perspective demonstrated on a display screen and including an orientation cue of the simulated data relative to the source data;

FIG. 5 is a representative diagram demonstrating modified image data having a simulated perspective demonstrated on a display screen and including an orientation cue of the simulated data relative to the source data;

FIG. 6A demonstrates an exemplary orientation cue indicating a portion of the source image data demonstrated by the modified image data;

FIG. 6B demonstrates an exemplary orientation cue indicating a portion of the source image data demonstrated by the modified image data;

FIG. 7A demonstrates an exemplary orientation cue indicating a portion of the source image data demonstrated by the modified image data;

FIG. 7B demonstrates an exemplary orientation cue indicating a portion of the source image data demonstrated by the modified image data; and

FIG. 8 is a modified block diagram demonstrating a surgical imaging system in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

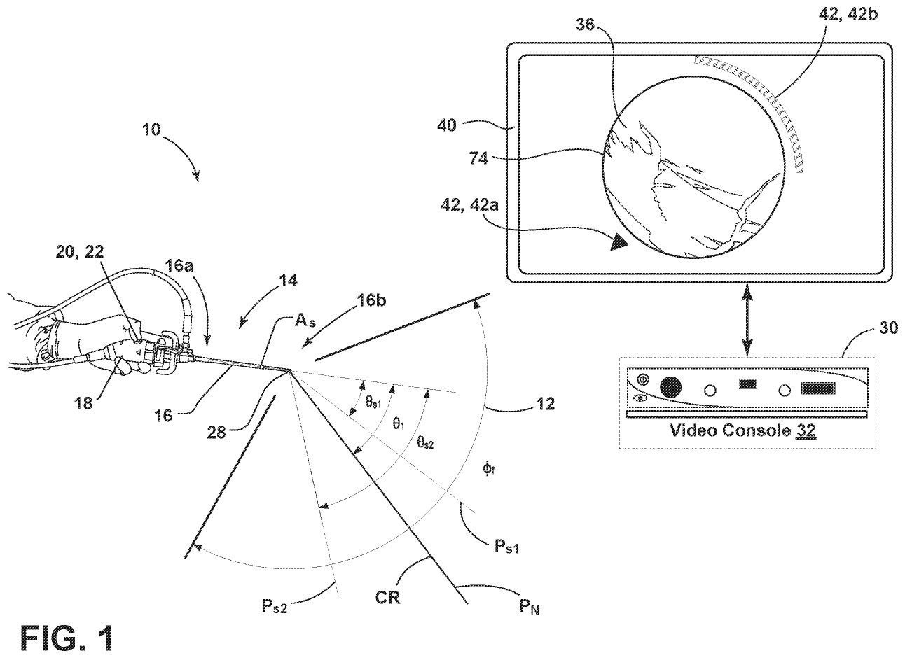

Referring to FIG. 1, the disclosure generally provides for a surgical imaging system 10 that may be implemented or in communication with a wide variety of surgical consoles, devices, and, particularly, cameras or scopes configured to capture image data in a surgical field of view 12. As shown, the system 10 may comprise a camera apparatus 14 that may include a scope 16 and camera body 18 configured to capture image data at a native perspective Pₙ relative to a distal end portion 16b of the scope 16. A proximal end portion 16a of the scope 16 may be connected to the camera body 18 and provide for a user interface 20 that may comprise one or more user inputs (e.g., buttons, switches, dials, etc.). In this configuration, an operator of the camera apparatus 14 may adjust and manipulate the field of view 12 captured by the scope 16 by manipulating and interacting with the camera body 18 and the user interface 20.

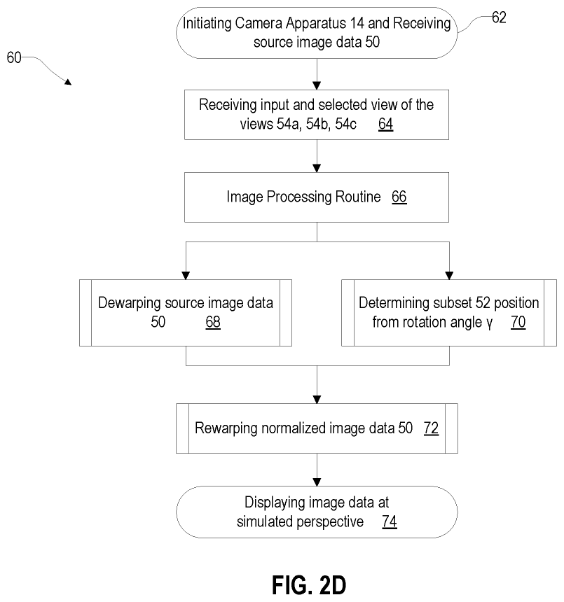

In various implementations, the scope 16 may comprise an optic element 28 in connection with the camera apparatus 14 at the distal end portion 16b. The optic element 28 may be aligned at an inclination angle θ that may define the native perspective Pₙ or a camera perspective along which an image sensor of the camera apparatus 14 captures source image data in the field of view 12. The image sensor may be positioned in the camera body 18 and optically coupled to the optic element 28 via an optical coupling (e.g., fiber-optic cable). In some implementations, the image sensor, control circuitry, and the optical element 28 may both be disposed in the distal end portion 16b of the scope 16 (e.g., a chip-on-tip camera). In such implementations, the camera body 18 as depicted may be omitted or incorporated to house circuitry associated with the operation of the image sensor and/or the user interface 20 and associated user engagement surface of the camera apparatus 14. In various implementations, as later exemplified by FIG. 3, the inclination angle θ defining the native perspective Pₙ may be configured to position a center ray CR of a plurality of light rays impinging upon the optic element 28 that passes, with little to no optic deflection, through the optic element 28 (e.g., a lens, prism, lens array, etc.). Accordingly, the camera apparatus 14 may be implemented in a variety of hardware configurations to provide the various features and operations discussed herein.

Referring still to FIG. 1, in various implementations, the imaging system 10 may comprise a controller 30 that may be incorporated in a video console 32. In operation, the controller 30 may receive source image data from an image sensor of the camera 18 depicting a scene representing light captured in the field of view 12 at the native perspective Pₙ, which may be defined by the inclination angle θ as previously discussed. In various implementations, the field of view 12 defined by the optic element 28 may correspond to a wide angle field, which may be referred to herein as a complete or full viewing angle ϕ_{f}. In various implementations, the full viewing angle ϕ_{f} may include viewing angles that may be greater than 110°, 120°, and in some implementations can exceed 140° or even 180°. In the instant example, the full viewing angle ϕ_{f} may correspond to a 140° field of view that may be captured at the native perspective Pₙ of approximately 45°. As discussed in the following examples, the controller 30 of the system 10 may provide for the generation of modified image data simulated to represent the field of view 12 captured at one or more simulated perspective angles (e.g., θₛ₁, θₛ₂, etc.) that may be offset relative to the inclination angle θ associated with the native perspective Pₙ.

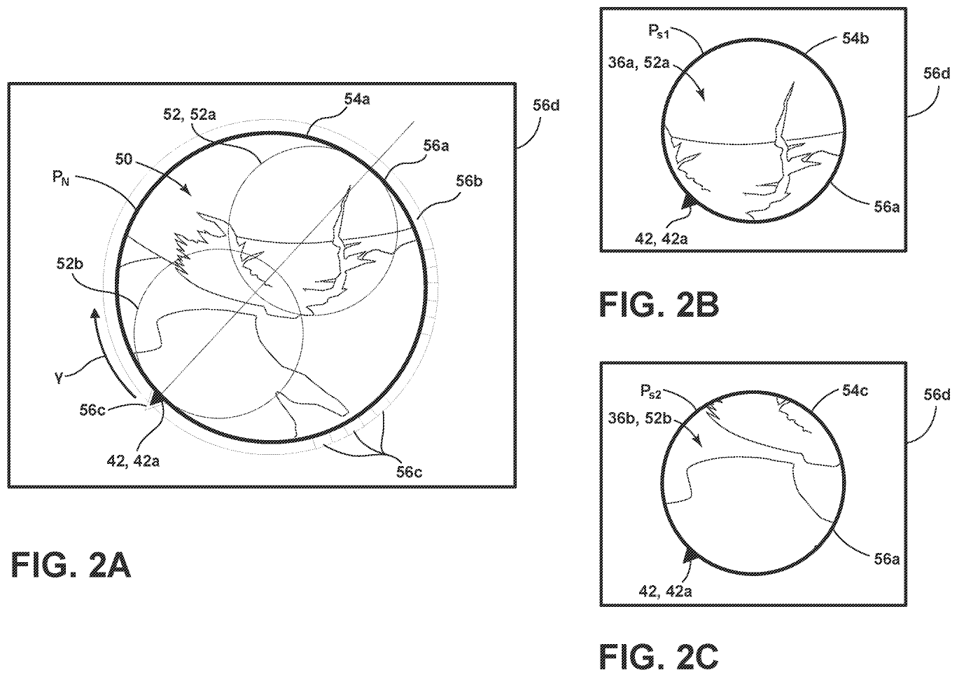

As previously discussed, the image sensor of the camera 18 may capture source image data 50 having a center ray CR aligned with the inclination angle θ along the native perspective Pₙ. In operation, the controller 30 may selectively generate the modified image data having a simulated perspective angle θₛ₁, θₛ₂ that may be offset from the inclination angle θ. For example, as discussed in further detail in reference to FIG. 2, the controller 30 may process the source image data depicting the full viewing angle ϕ_{f} of the field of view 12 to generate modified image data 36 having a simulated perspective angle θₛ₁ offset from the inclination angle θ to provide a simulated perspective Pₛ₁. In the example of the first simulated perspective Pₛ₁, the simulated perspective angle θₛ₁ may be offset by approximately 15° to provide a first simulated perspective Pₛ₁ at an angle of 30°. Similarly, the controller 30 may remap the source image data (e.g., the pixel values associated with the image data) to generate the modified image data 36 representing a second simulated perspective Pₛ₂ that may be remapped to represent a second simulated perspective angle θₛ₂ of approximately 70°. As discussed in the following examples, the simulated perspectives Pₛ₁ and Pₛ₂ and corresponding simulated perspective angles θₛ₁, θₛ₂ may be selectively activated to generate and output the corresponding modified image data 36 to a display screen 40. Though referred to in reference to specific angular values for the simulated perspective angles θₛ₁, θₛ₂; the simulated perspective angles may range from corresponding inclination angles ranging from approximately 0° to 90° or offsets from the native perspective Pₙ ranging from approximately −30° to 45°.

Before moving on to discuss the generation of the modified image data in further detail, the operation of the video console 32 may provide for one or more visual cues 42 that may assist a user in visually recognizing a relationship of the scope 16 to the image data depicted on the display screen 40 via an orientation cue 42a. Additionally, the visual cues 42 may include a relative position cue 42b that may indicate a subset of the source image data extending over the full viewing angle ϕ_{f} represented by the modified image data 36. In the example shown, the relative position cue 42b or perspective cue may identify a region of the source image data within the full viewing angle ϕ_{f} that may be available for viewing on the display relative to a subset of the field of view 12 that may be depicted by the modified image data 36.

Referring now to FIGS. 2A-2C, examples of the modified image data 36 (e.g., first modified image data 36a, second modified image data 36b) are demonstrated relative to source image data 50 depicting the full viewing angle ϕ_{f} of the field of view 12. As discussed in various examples, the disclosure may provide for the display of the modified image data 36 as one or more subsets 52 that may depict a detailed portion of the source image data 50. In the example shown, the subsets 52 may comprise a first subset 52a and a second subset 52b of the source image data 50 that may be modified and/or remapped by the controller 30 to generate the first modified image data 36a and the second modified image data 36b, respectively. As demonstrated in FIG. 2A, the subsets 52 of the source image data 50 utilized to generate the modified image data 36 may consistently be aligned with a rotation angle γ of the scope 16 relative to the camera 18. In this configuration, the inclination angle θ as well as the simulated perspective angles θₛ₁, θₛ₂ may be consistently aligned with the rotation angle γ of the scope 16 and communicated to the user via one or more of the visual cues 42. In this way, the imaging system 10 may provide for the selective output and display of the modified image data 36 representative either of the simulated perspective angles Pₛ as well as the source image data 50. Such selective display of the image data associated with the field of view 12 in the subsets 52 and the various perspectives Pₙ, Pₛ₁, Pₛ₂ may provide for improved flexibility in operation of the camera apparatus 14.

In operation, the controller 30 may selectively output the source image data 50 as well as the first or second modified image data 36a, 36b to demonstrate either the full field of view 12 over the full viewing angle ϕ_{f} or the subsets 52a, 52b corresponding to the simulated scope or perspective angles θₛ₁, θₛ₂. For example, the controller 30 may cycle through a first view 54a demonstrating the full viewing angle ϕ_{f}, a second view 54b demonstrating the source image data 50 in a region corresponding to the first simulated perspective angle θₛ₁, and a third view 54c demonstrating the source image data 50 in a region corresponding to the second simulated perspective angle θₛ₁. Each of the views 54a, 54b, 54c may be selectively output to the display 40 in response to an input to the user interface 20 of the camera apparatus 14. In this way, the controller 30 may present the first view 54a demonstrating a wide viewing field encompassing both the first subset 52a and the second subset 52b. Additionally, the controller 30 may selectively generate and output the second view 54b or the third view 54c, which correspond to subsets 52 of the first view 54a.

Still referring to FIGS. 2A-2C, the generation and display of the views 54 may include various image processing, masking, cropping, or similar procedures. In various examples, the generation of the visual cues 42 (e.g., the orientation cue 42a and relative position cue 42b) may be included in the generation of a virtual mask 56a extending about the perimeter of each of the views 54a, 54b, 54c. The virtual mask 56a may be superimposed over a field stop mask that may define the perimeter of the source image data 50. For example, in various cases, a field stop mask 56b associated with the scope 16 of the camera apparatus 14 may include a variety of features 56c (e.g., shapes, pointers, irregularities, etc.) that may indicate the rotation angle γ of the scope 16 relative to the camera body 18 and correspond to additional variations about the perimeter of the source image data 50. These variations and features 56c associated with the field stop mask 56b may be distracting to a user of the system 10. Accordingly, the controller 30 may generate the virtual mask about the perimeter of the source image data 50 creating a smooth perimeter boundary extending from an edge of the source image data 50 or modified image data 36a, 36b to the edges of a window 56d output to the display 40.

In addition to the generation of the virtual mask 56a about the source image data 50, the controller may similarly generate the virtual mask 56a about the views 54b, 54c demonstrating the modified image data 36a, 36b corresponding to the subsets 52a, 52b. For each of the views 54a, 54b, 54c, the virtual mask 56a may comprise the visual cues 42 (e.g., the orientation cue 42a and relative position cue 42b). In operation, the controller 30 may detect one or more of the features 56c in the source image data 50 to detect the rotation angle γ of the scope 16. In this way, the controller 30 may identify the location about the perimeter of the source image data 50 to position the visual cues 42 to accurately identify the rotation angle γ. Similar to the source image data 50, the virtual mask 56a may be generated and applied to frame and enclose the subsets 52 corresponding to the modified image data 36 in each of the second view 54b and the third view 54c. Accordingly, the controller 30 may selectively output each of the views 54a, 54b, 54c, and apply the virtual mask 56a and/or the visual cues 42 to assist in the operation of the system 10.

To generate the view 54a, 54b, 54c, the controller 30 may further apply one or more image processing techniques, filters, and/or algorithms, referred to herein as image correction algorithms, to improve the lighting, contrast, etc. of the source image data 50. When outputting the source image data 50, the controller 30 may process the image data corresponding to the field of view 12 over the full viewing angle ϕ_{f}. Additionally, when applying the image correction algorithms to the second view 54b and the third view 54c, the controller 30 may limit a range of the image correction algorithm to the corresponding first subset 52a or second subset 52b. For example, when applying an auto-exposure algorithm to generate the second view 54b, the controller 30 may limit the image data processed from the source data 50 to the pixels or information located within the first subset 52a. In this way, the image correction algorithms and/or filters applied to generate the second view 54b may be limited to the range and attributes included in the first subset 52a, such that the associated lighting and features are optimized to the subset 52a rather than the entirety of the source image data 50.

Referring now to FIGS. 2A-2D, a flow chart is shown demonstrating an exemplary method 60 for generating the image data demonstrated in the subsets 52a, 52b. As provided in the following steps, the controller 30 may process the image data through a procedure that includes dewarping the source image data 50, subsampling the portion of the image data corresponding to the rotation angle γ, and rewarping the image data to generate the modified image data 36. As previously described, the modified image data 36 may correspond to one of the simulated perspectives Pₛ introduced in FIG. 1. As discussed in further detail in reference to FIGS. 3A-3C, the generation of the modified image data 36 may generally include a process of digitally manipulating the source image data 50 to remove distortions associated with the field of view 12 over the full viewing angle ϕ_{f} and generate normalized image data. Additionally, the subsets 52a, 52b may be sampled from the normalized source data and further warped or manipulated to generate the modified image data 36. In this way, the modified image data 36 may be displayed to mimic one of the simulated perspectives Pₛ.

As discussed herein, image warping may include various steps that may modify the positions or proportions of the source image data 50 via coordinate or pixel mapping or various forms of geometric transformation. For example, the source image data may be distorted, skewed, rotated, and/or translated to simulate visual aspects or characteristics of the simulated perspectives Pₛ. The warping or dewarping algorithms described herein may include forward warping, inverse warping, spline warping, mesh warping, etc. as well as selective magnification, smoothing, and other image processing methods to normalize distortions in the source image data 50 and/or simulate characteristic distortions or features of the simulated perspectives Ps. In this way, the controller 30 may selectively generate the image data at the simulated perspectives Ps to suit a variety of applications.

In operation, the method 60 may be initiated in response to the activation of the camera apparatus 14 and receiving the source image data 50 (62). Once activated, the controller 30 may begin processing the source image data 50 to demonstrate one of the views 54a, 54b, 54c (64). In following example, the selected view will be described in reference to the first view 54a demonstrating the full viewing angle ϕ_{f}. However, it shall be understood that the view 54, upon initialization, may correspond to any of the views 54a, 54b, 54c. In the example shown, the input to the user interface 20 may cause the controller 30 to generate and display the first subset 52a of the source image data 50 via an image processing routine (66). In operation, the image processing routine 66 may process the source image data 50 on a frame-by-frame or selective basis by applying a dewarping algorithm (68). The dewarping algorithm may correct for distortions or otherwise normalize the source image data 50 to correct for any irregularities or variations in magnification of one or more lenses or optics used to capture the source image data 50 at the full viewing angle ϕ_{f}. In this way, the controller 30 may generate normalized image data for additional processing to generate the modified image data 36 at a simulated perspectives Pₛ.

Concurrent to or in sequence with the generation of the normalized image data in step 68, the controller 30 may determine the portion of the source image data 50 from which to sample the first subset 52a based on the rotation angle γ of the scope 16 (70). As previously discussed, the rotation angle γ of the scope 16 may be identified in response to one or more of the features 56c in the source image data 50, which may remain in a fixed relationship relative to the camera 18 as the scope 16 is rotated. Once the rotation angle γ of the scope 16 is identified based on the features 56c, the controller 30 may continue the image processing routine 66 by selecting the subset 52a of the source image data 50 in step 70. As shown in FIG. 2A, the subset 52a may be positioned tangential to a perimeter of the source image data 50 or otherwise offset from a center of the source image data 50 to simulate a viewing angle of the scope 16 at the one of the simulated perspective angles θₛ₁, θₛ₂. Following the selection of the first subset 52a, the image processing routine 66 may continue by processing the first subset 52a with a rewarping algorithm as further discussed in reference to FIGS. 3A-3C (72). For example, the rewarping algorithm may process the modified image data in the subset 52a to simulate the native magnification, zoom, and/or irregularities associated with the selected one of the simulated perspective angles θₛ₁, θₛ₂. The method 60 may continue to process the image data for display as described in steps 62-74 throughout the operation of the camera apparatus 14.

In operation, the method 60 may operate as a continuous image feed via an image processing pipeline. Accordingly, responsive to the change in the rotation angle γ of the scope 16, the subset 52 of the source image data 50 may be reselected or updated to correspond to the portion of the source image data 50 aligned with the rotation angle γ. As represented by the rotation arrows 61 in FIG. 2A, the subset 52 of the source image data 50 presented in each of the image frames may be updated based on the rotation angle γ of the scope 16 at the time of the capture of each frame. Accordingly, the rotation of the scope 16 relative to the camera apparatus 14 may result in the gradual rotation of the position of the subset 52 within the source image data 50 and the corresponding generation of the modified image data 36 for display. In the example shown, the position of the subset 52 is updated along the positions represented by the rotation arrows 61 over a period of time corresponding to the change in the rotation angle γ from the first subset 52a to the second subset 52b.

In some implementations, the source image data 50 may be selectively displayed in various optional formats for each of the views 54a, 54b, 54c. For example, as described, the source image data 50 may be dewarped, normalized, or flattened as discussed in step 68 to correct for distortions, irregularities, or variations in magnification of the optic element 28. In some cases, rather than displaying the source image data 50 at the native Pₙ or the modified image data 36 at the simulated perspectives ₛ, the controller 30 may be configured to display the normalized image data for any one of the views 54a, 54b, 54c. Additionally, the modified image data 36 may be modified in other ways (e.g., difference levels of distortion, magnification, etc.) to present the modified image data 36 in a custom display format. In general, the custom display format may be modified using similar techniques used to generate the simulated perspectives Pₛ. However, the custom display format may adjust, distort, and/or magnify the normalized image data to conform to a user preference or various preconfigured perspectives and views. Accordingly, the controller 30 may be configured to selectively display the normalized image data or the modified image data with the custom display format for each of the views 54 or corresponding subsets 52 of the field of view 12 captured by the camera apparatus 14.

Referring now to FIGS. 3A-3C, exemplary methods and processing steps utilized by the controller 30 to generate the modified image data 36 are discussed in further detail. As previously discussed, the source image data 50 may be captured by the camera apparatus 14 represented as an image sensor 90. As previously discussed, the source image data 50 may be captured at the native perspective Pₙ defining a center ray CR that passes undeflected through the center or focal center of the optic element 28 to the image sensor 90. In some examples, the center ray CR may be detected by the controller 30 by identifying or calculating the actual position of the optic element 28, which may vary based on tolerances and alignments associated with manufacturing and assembly. By detecting the actual position of the center ray CR, the controller 30 may consistently generate the simulated perspectives Pₛ regardless of variations among scopes 16. The detection of the actual position of the center ray CR based on the position through which light passes undeflected through the optic element may further be beneficial in positioning the virtual mask 56a, such that the virtual mask 56a is radially centered about the center ray CR of the native perspective Pₙ and modified center rays MCRs of the simulated perspectives Pₛ.

To generate the modified image data 36 at the simulated perspectives Pₛ, the controller 30 may offset the center ray CR to a modified center ray MCR and remap each of the plurality of rays 92 impinging upon the optic element 28 at the native perspective Pₙ to correspond to an angular offset of the modified center ray MCR. As demonstrated in the examples shown in FIGS. 3B and 3C, the plurality of rays 92 associated with the native perspective Pₙ may be modified to align with each of the simulated perspectives Pₛ along the modified center ray MCR. In this way, the modified image data 36 corresponding to the subsets 52 from the source image data 50 may not only correspond to cropped and magnified portions of the source image data 50, but may further provide for a simulated representation of the source image data 50 as though it were captured along one of the simulated perspectives Pₛ. As described herein, the focal center may correspond to a central portion of the field of view 12 that may not correspond to a geometric center of the lens. For example, the focal center may be offset from the geometric center to limit distortion or provide for the use of asymmetric lenses for various applications.

In the example shown in FIG. 3B, the first modified image data 36a may be generated from the source image data 50 corresponding to the first subset 52a by offsetting a first modified center ray MCR₁ to align with the first simulated perspective angle θₛ₁. As shown, θₛ₁ is represented by the offset from the native perspective Pₙ to the first simulated perspective angle θₛ₁ as Δθ₁. The offset Δθ₁ may be representative of the computational offset required to be applied by the controller 30 to remap each of a plurality of modified rays 94 represented by the modified image data 36 to be aligned with the first modified center ray MCR₁ and distributed thereabout to remap the source image data 50 to appear to have been captured along the first simulated perspective Pₛ₁.

Similar to the procedure discussed in reference to FIG. 3B, FIG. 3C demonstrates a procedure for generating the second modified image data 36b along a second modified center ray MCR₂. The second simulated perspective Pₛ₂ is again represented based on the angular offset Δθ₂ between the native perspective Pₙ and the second simulated perspective Pₛ₂. In operation, the controller 30 may remap the source image data 50 associated with the second subset 52b to align with the second modified center ray MCR₂. Additionally, the plurality of rays 92 associated with the native perspective Pₙ in the second subset 52b may be remapped to align with the second modified center ray MCR₂ to generate the plurality of modified rays 94. In this way, the controller 30 may modify the source image data 50 to appear as though it was captured along the second simulated perspective Pₛ₂.

To more clearly describe the computational aspects associated with the generation of the modified image data 36, a general discussion is now provided in reference to FIGS. 3A-3C.

Conceptually, the plurality of rays 92 demonstrated in FIG. 3A may correspond to a unit sphere defining the center ray CR passing centrally through the optic element 28, such that the center ray CR passes undeflected therethrough. As depicted in FIGS. 3B and 3C, the lens or optic element 28 is presented in a first offset orientation 28a and a second offset orientation 28b aligned with the first simulated perspective Pₛ₁ and the second simulated perspective Pₛ₂, respectively, denoting the simulated posture of the lens associated with each of the perspectives Pₛ. Additionally, the plurality of modified rays 94 are shown representing the alignment of the corresponding simulated light and resulting modified image data 36 aligned with each of the modified center rays MCR₁, MCR₂. In operation, the controller 30 may modify the corresponding source image data 50 in the subsets 52 corresponding to each of the plurality of modified rays 94 to simulate the field of view 12 captured along each of the simulated perspectives Pₛ₁, Pₛ₂.

More specifically, in an exemplary implementation, the pixel values associated with the source image data 50 may be modified via a complex series of spherical, polar, and cartesian calculations for each of the plurality of modified rays 94 associated with the simulated perspectives Pₛ. For every cartesian raster pixel required to create the modified image data 36 at the simulated perspective Pₛ, the corresponding location of the pixel relative to the modified center ray MCR may be calculated in two-dimensional polar coordinates. This location may then be scaled according to the proportions of the subset 52 forming the portion of the source image data 50 demonstrated in the modified image data 36. A mapping function may then be applied to the polar coordinates of each of the pixels to relocate the pixels according to the transformed view associated with the simulated perspective angle Pₛ. Such a transformation may be dependent on the specific properties of the optic element 28. For example, if the operation of the optic element 28 corresponds to a tangent mapping function, an arc-tangent function may be applied to the pixel locations and polar coordinates relative to the modified center ray MCR to flatten the image. This operation may be similarly applied to an undistorted method utilized to flatten image data. Additionally, similar functions may be applied to achieve the same effect for various types of lens and corresponding lens mapping functions.

Once each of the pixels associated with the modified image data 36 is transformed, a two-dimensional polar origin may be assigned to align with the modified center ray MCR of the simulated perspective Pₛ in three-dimensional spherical coordinates. With the simulated perspective Pₛ assigned or assumed for the modified image data 36, the corresponding pixel data may be calculated corresponding to each of the plurality of modified rays 94 aligned with the modified center ray MCR. The pixel data corresponding to the plurality of modified rays 94 may then be mapped back to two-dimensional polar coordinates against the source image data 50 with a polar origin aligned with the center ray CR of the optic element 28. With the pixel data corresponding to the modified rays 94 normalized in two-dimensional polar coordinates, a lens distortion correction may further be applied to adjust the representation of the corresponding pixel data that may result from the distortions associated with the optic element 28. Following the lens distortion correction, the cartesian X-Y location of the source pixels for corresponding portions of the source image data 50 can be calculated by deformalizing the location by a focal length of the optic element 28 and converting from polar to cartesian coordinates for each of the source pixels from the source image data 50. As the location of the pixels desired to generate the modified image data 36 may fall between pixel locations in the source image data 50, the pixel values associated with the source image data 50 may be interpolated to output the modified image data 36 via various interpolation methods (e.g., bi-linear interpolation of the four nearest pixel neighbors). Repeating this process for every pixel in the corresponding subset 52 may provide for the remapped pixel information corresponding to the modified image data at the simulated perspective Pₛ. Though specific computational operations are described in the aforementioned example, alternative methods may be implemented to generate the simulated perspectives without departing from the spirit or scope of the disclosure.

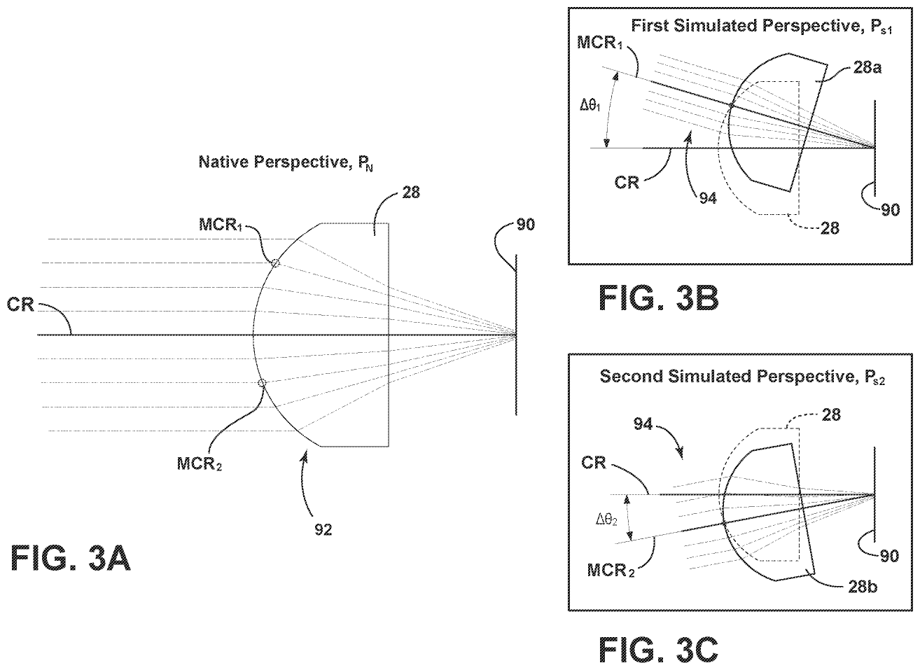

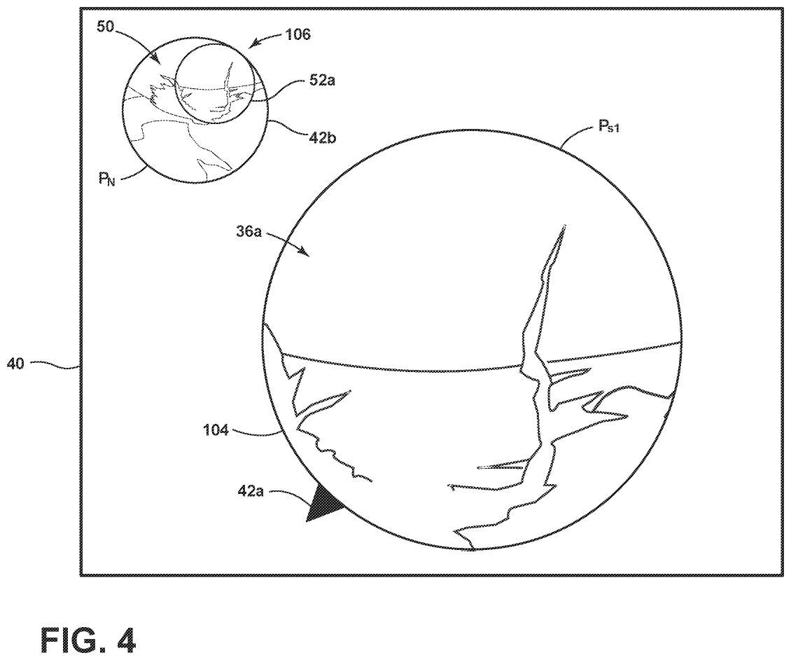

With the modified image data 36 generated by the controller 30, the video console 32 may output the modified image data 36 to the display screen 40 in a variety of ways. Various examples of display configurations and methods associated with the source image data 50 and the modified image data 36 are now discussed in reference to FIGS. 4-7. As shown in FIGS. 4 and 5, the modified image data 36 may be demonstrated contemporaneously on the display screen 40 with one or more visual cues 42. In the example shown, the visual cues 42 include an orientation cue 42a that may identify the rotation angle γ of the scope 16. Additionally, a relative position cue 42b may be included that identifies a positional relationship between the subset 52 of the source image data 50 and the modified image data 36 displayed. As shown in FIG. 4, the first subset 52a may be demonstrated as a marker, outline, and/or overlay superimposed over the source image data 50 depicted on the display screen 40. Similarly, in FIG. 5, the relative position cue 42b may demonstrate the location of the second subset 52b within the source image data 50 via a similar marker, outline, and/or overlay. In this configuration, the imaging system 10 may provide for intuitive visual cues 42 ensuring that the selected depiction of the source image data 50 and/or the modified image data 36 may be readily determined by viewing the display screen 40.

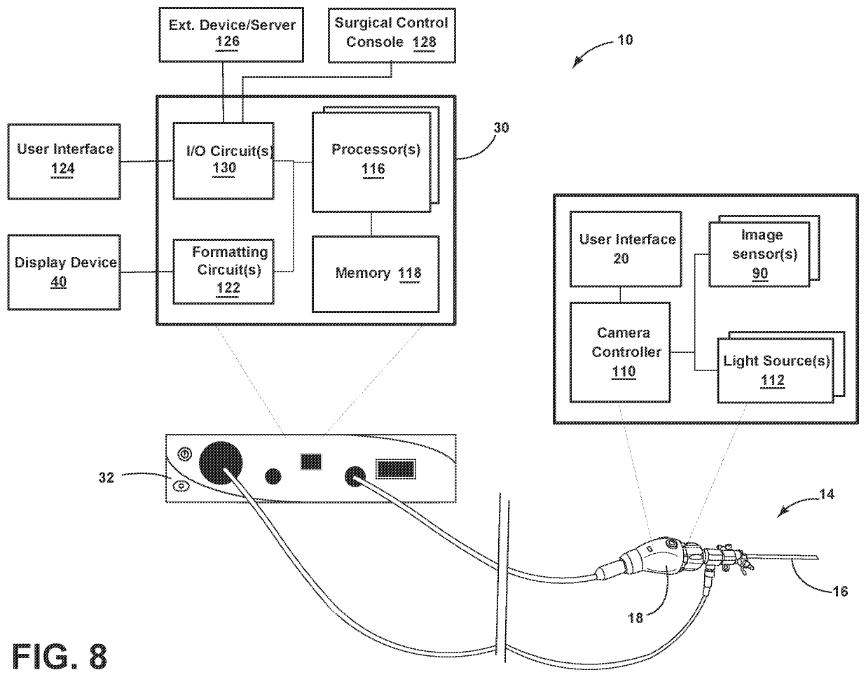

As previously described, the mapping function may be applied to each of the pixels to relocate the pixels according to the transformed view associated with the simulated perspective angle Ps. In some cases, the system 10 may store calibration data in memory 118 (FIG. 8) that may define the mapping function based on locations and transformation measures for one of more types, classes, or specific models of scope associated with the simulated perspective angles Ps. Further, while the mapping function may be applied generally to map the simulated perspective angles Ps from the native perspective Pₙ, the controller 30 of the system 10 may further identify the specific model of the scope 16 and corresponding calibration data. For example, based on the detected model information for the scope 16, the controller 30 may identify whether the scope 16 is compatible to generate image data at one or more of the simulated perspective angles Ps. Additionally, the controller 30 may access model-specific calibration data for the model of the scope 16 identified. In response to the model-specific calibration data, the scope 16 may update the mapping function based on the native calibration (e.g., perspective, magnification, distortion, etc.) for the specific model of the scope to update the mapping function to accurately generate the one or more simulated perspective angles Ps from the native perspective Pₙ.

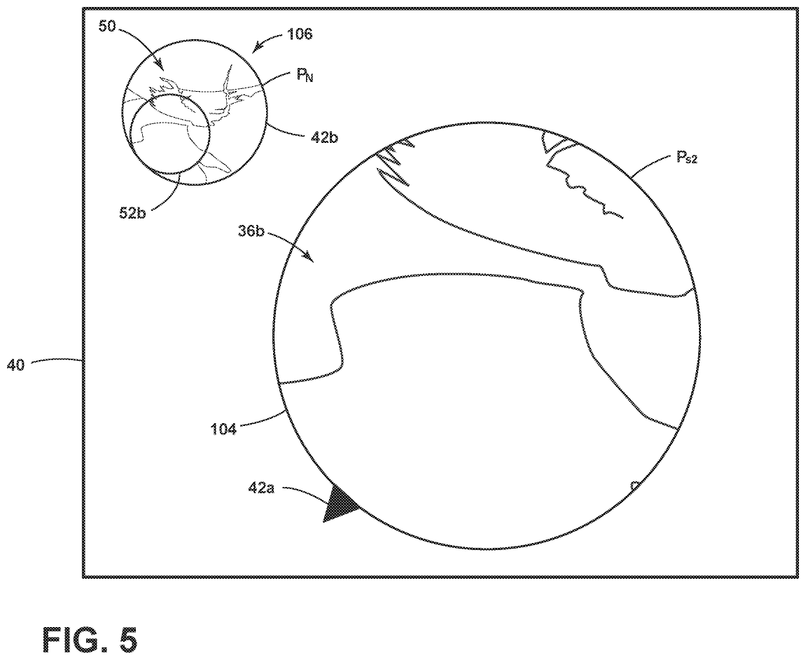

As shown in FIGS. 6A and 6B, the orientation cue 42a is similarly demonstrated. In these examples, the relative position cue 42b may correspond to a graphic 100 that may be positioned along a perimeter of the depiction of the modified image data 36 corresponding to a portion of the field of view that may be expanded from the subset 52 depicted in the modified image data 36. For example, the graphic 100 may correspond to a band enclosing a portion of the perimeter 102 of a viewing window 104. The band formed by the graphic 100 may extend about the perimeter 102 along a region where the source image data 50 may be expanded to increase the scope demonstrated within the viewing window 104. For example, if the first simulated perspective Pₛ₁ is demonstrated within the viewing window 104, the controller 30 may position the band formed by the graphic 100 to indicate that a remainder of the source image data 50 may be expanded along the perimeter 102 in the direction of the graphic 100. For a visual representation of the direction of expansion identified by the graphics 100 in FIGS. 6A and 6B, the corresponding simulated perspectives Pₛ₁ and Pₛ₂ are shown in FIG. 2A relative to the full viewing angle ϕ_{f} of the field of view 12 captured in the source image data 50. Accordingly, the graphics 100 demonstrated in FIGS. 6A and 6B may provide visual indications of directions for regions of the field of view 12 that may be expanded from the subsets 52 depicted in the simulated perspectives Pₛ in various examples.

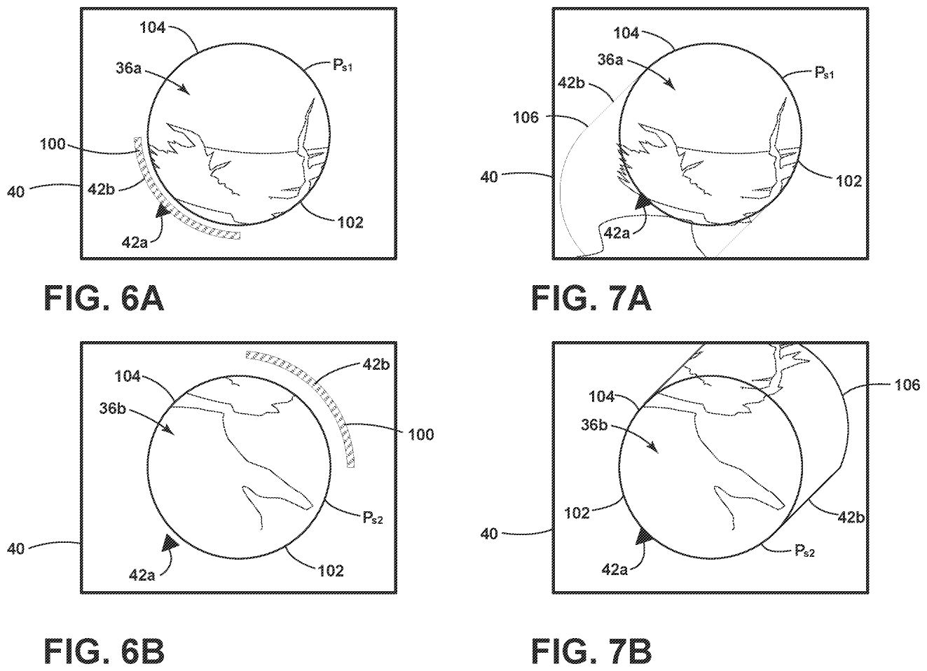

As shown in FIGS. 7A and 7B, the relative position cues 42b may correspond to preview windows 106 that may be positioned adjacent to the viewing window 104 or outside the perimeter 102 depicting portions of the source image data 50 outside the viewing window 104. Similar to the previous examples discussed, the relative position cues 42b provided in the examples of FIGS. 7A and 7B may extend along portions of the perimeter 102 of the viewing window 104 where the field of view 12 captured by the source image data 50 over the full viewing angle ϕ_{f} may expand outside of the subset 52 depicted within the viewing window 104. The depiction of such visual cues 42 may ensure that a user of the imaging system 10 may not only be aware of the view presented as corresponding to one of the simulated perspectives Pₛ provided by the modified image data 36 or the source image data 50 but may also ensure that the availability and content associated with the views not presently presented on the display screen 40 may be intuitively determined by the user.

Referring now to FIG. 8, a block diagram of the imaging system 10 is shown. As discussed throughout the disclosure, the system 10 may comprise the camera apparatus 14 in communication with the controller 30. The camera apparatus 14 may comprise a camera controller 110, a light source 112, the image sensor 90, and the user interface 20. In various implementations, the camera apparatus 14 may correspond to an endoscope, laparoscope, arthroscope, etc. formed by the scope 16 in the form of an elongated probe comprising a narrow distal end 16b suited to various noninvasive surgical techniques. For example, the distal end 16b may include a diameter of less than 2 mm. As demonstrated, the camera apparatus 14 may be in communication with the controller 30 via a communication interface. Though shown connected via a conductive connection, the communication interface may correspond to a wireless communication interface operating via one or more wireless communication protocols (e.g., Wi-Fi, 802.11 b/g/n, etc.).

The light source 112 may correspond various light emitters configured to generate light in the visible range and/or the near infrared range. In various implementations, the light source 112 may include light emitting diodes (LEDs), laser diodes, or other lighting technologies.

The image sensor 90 or image sensor may correspond to various sensors and configurations comprising, for example, charge-coupled devices (CCD) sensors, complementary metal-oxide semiconductor (CMOS) sensors, or similar sensor technologies. In various implementations, the camera controller 110 may correspond to a control circuit configured to control the operation of image sensor 90 and the light source 112 as well as process and/or communicate the source image data 50 to the controller 30 or system controller. Additionally, the camera controller 110 may be in communication with the user interface 20, which may include one or more input devices, indicators, displays, etc. The user interface 20 may provide for the control of the camera apparatus 14 including the activation of one or more routines as discussed herein. The camera controller 110 may be implemented by various forms of controllers, microcontrollers, application-specific integrated controllers (ASICs), and/or various control circuits or combinations.

The controller 30 or imaging controller may comprise a processor 116 and a memory 118. The processor 116 may include one or more digital processing devices including, for example, a central processing unit (CPU) with one or more processing cores, a graphics processing unit (GPU), digital signal processors (DSPs), field programmable gate arrays (FPGAs), application specific integrated circuits (ASICs) and the like. In some configurations multiple processing devices are combined into a System on a Chip (SoC) configuration while in other configurations the processing devices may correspond to discrete components. In operation, the processor 116 executes program instructions stored in the memory 118 to perform the operations described herein.

The memory 118 may comprise one or more data storage devices including, for example, magnetic or solid state drives and random access memory (RAM) devices that store digital data. The memory 118 may include one or more stored program instructions, object detection templates, image processing algorithms, etc. As shown, the memory 118 may comprise one or more modules that may include instructions to process the source image data 50 and generate the modified image data 36. For example, the processor 116 may access instructions in the memory modules to perform various processing tasks on the image data including preprocessing, filtering, masking, cropping, and various enhancement techniques to improve visibility and generation of the simulated perspectives Pₛ₁.

In some implementations, the controller 30 may correspond to a display controller. In such applications, the controller 30 may include one or more formatting circuits 122, which may process the image data received from the camera apparatus 14, communicate with the processor 116, and process the image data according to one or more of the operating methods discussed herein. The formatting circuits 122 may include one or more signal processing circuits, analog-to-digital converters, digital-to-analog converters, etc. The display controller may comprise a user interface 124, which may be in the form of an integrated interface (e.g., a touchscreen, input buttons, an electronic display, etc.) or may be implemented by one or more connected input devices (e.g., a tablet), peripheral devices (e.g., keyboard, mouse, etc.), foot pedals, remote switches, etc.

As shown, the controller 30 is also in communication with an external device or server 126, which may correspond to a network, local or cloud-based server, device hub, central controller, or various devices that may be in communication with the controller 30 and, more generally, the imaging system 10 via one or more wired (e.g., Ethernet) or wireless communication (e.g., Wi-Fi, 802.11 b/g/n, etc.) protocols. For example, the controller 30 may receive updates to the various modules and routines as well as communicate sample image data from the camera apparatus 14 to a remote server for improved operation, diagnostics, and updates to the imaging system 10. The user interface 124, the external server 126, and/or a surgical control console 128 may be in communication with the controller 30 via one or more I/O circuits 130. The I/O circuits 130 may support various communication protocols including, but not limited to, Ethernet/IP, TCP/IP, Universal Serial Bus, Profibus, Profinet, Modbus, serial communications, etc.

According to some aspects of the disclosure, a surgical imaging system comprises a scope including an optic element aligned at a first inclination angle defining a native perspective, an image sensor configured to capture source image data in a field of view transmitted through the optic element, and a controller. The control is configured to control the capture of source image data at the native perspective at the first inclination angle; select a first subset of the source image including a first portion of the field of view simulating a second inclination angle; and selectively output modified source image data simulating the second inclination angle to a display screen.

According to various aspects, the disclosure may implement one or more of the following features or configurations in various combinations:
the first subset is offset from a focal center of the field of view of the source image data based on a difference between the first inclination angle and the second inclination angle;
the controller is further configured to detect one or more features in the source image data indicating a rotation of the scope relative to a camera body, wherein the selection of the first subset is responsive to the rotation of the scope;
the controller is further configured to dewarp the first subset of the source image data generating normalized image data and warp or modify the normalized image data in the first subset generating the modified source image data simulating a second inclination angle;
the dewarping of the first subset is corrected for one or more distortions or magnifications of the native perspective at the inclination angle;
the warping of the normalized image data is distorted, simulating a magnification or distortion at the second inclination angle;
the controller is further configured to generate a simulated mask enclosed about a perimeter of the first image, wherein the simulated mask includes an orientation cue identifying a direction of the rotation relative to the first image data demonstrating the first portion of the field of view simulating a second inclination angle;
the controller is configured to generate the modified source image data by generating a virtual mask superimposed over a field stop mask of the scope about the field of view;
the controller is further configured to selectively generate second image data demonstrating a second subset of the source image data including a second portion of the field of view simulating a third inclination angle;
the first inclination angle is approximately 45° offset from a scope axis of the scope;
the controller is further configured to selectively output the source data, the first image data, and the second image data to the display screen;
the second inclination angle is 30° and the third inclination angle is 70°;
the one or more features comprise a field stop mask of the scope demonstrated in the source image data;
the generation of the first image data comprises applying an image correction algorithm, wherein the controller is further configured to limit a range of the image correction algorithm to the first subset of the source image data for the first image data;
the image correction algorithm comprises at least one of an auto-exposure algorithm and a high dynamic range algorithm; and/or
a method for operating the surgical imaging system.

According to another aspect of the disclosure, a surgical imaging system comprises a scope including an optic element aligned at an inclination angle defining a native perspective;

an image sensor configured to capture source image data in a field of view transmitted through the optic element; and a controller. The controller is configured to control the capture of source image data at the native perspective at the inclination angle, selectively generate modified image data from the source image data having a simulated view relative to the field of view at the native perspective, wherein the simulated view is modified to represent a simulated perspective different than the native perspective, and output the modified image data to a display screen.

According to various aspects, the disclosure may implement one or more of the following features or configurations in various combinations:
the native perspective is offset approximately 45° from a scope axis of the scope;
the simulated view comprises a simulated perspective angle that ranges from approximately −30° to 45° from the native perspective;
the source image data comprises a set of pixels and the simulated perspective is generated by remapping light rays aligned with the set of pixels in the source image with an offset center ray aligned with the simulated perspective angle;
the source image data comprises a set of image data and the modified image data forms a subset of the source image data, wherein the subset of the modified image data is processed to adjust a lighting or exposure and wherein the processing of the subset of the source image data is processed within the simulated view masking a remainder of the source image data from the adjustment of the lighting or exposure;
the source image data comprises a unit sphere of light rays captured in the field of view about a center ray of the lens;
the native perspective defines the center ray received centrally within the field of view and the center ray passes undeflected through a focal center of the lens;
the modified image data is generated for the simulated perspective at a modified center ray offset from the focal center of the lens;
the modified image data is simulated to appear as though the modified center ray passes undeflected through the lens within the field of view;
the modified image data is calculated based on a relative position of a plurality of modified rays distributed about the modified center ray;
the controller is further configured to detect an actual position of a center ray through the lens defining a focal center of the lens;
the controller is further configured to generate a virtual mask centered radially about the actual position of the center ray;
the modified image data is generated based on the actual position of the center ray detected by the controller;
the modified image data is generated by remapping the source image data to conform to the simulated perspective by adjusting pixel values in the field of view to correspond to rays of light impinging on the lens at an adjusted angle offset based on a simulated perspective angle;
the source image data is captured over a plurality of source image frames and the modified image data is generated as modified image frames forming a modified image stream;
the controller is further configured to selectively output the source image data or the at least one modified image data in response to an input from a device in communication with the controller;
the user interface comprises at least one of a camera interface in connection with the scope, an auxiliary input accessory (e.g., foot pedal, hand switch, etc.), a tablet, a computer terminal, a surgical tool or tool control console (e.g., shaver console, ablation console, pump, etc.);
the modified image data is selectively generated having a simulated perspective angle of 30° and 70°, and the native perspective is aligned with the inclination angle of 45°;
the source image data is captured over a full viewing angle of the field of view and the modified image data depicts a subset of the source image data;
the controller is further configured to generate a visual cue indicating a region within the source image data depicted by the modified image data;
the visual cue comprises a graphic presented contemporaneous to the modified image data and indicating the region within the source image data where the subset is located;
the graphic comprises a symbol identifying a positional relationship between the subset and the source image data; and/or
the graphic comprises a marker, outline, and/or overlay superimposed over the source image data demonstrating a region of the subset within the source image data.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A surgical imaging system comprising:
a scope comprising an optic element aligned at a first inclination angle defining a native perspective;
an image sensor configured to capture source image data in a field of view transmitted through the optic element; and
a controller configured to:
control the capture of source image data at the native perspective at the first inclination angle;
select a first subset of the source image including a first portion of the field of view simulating a second inclination angle; and
selectively output modified source image data simulating the second inclination angle to a display screen.

2. The imaging system according to claim 1, wherein the first subset is offset from a focal center of the field of view of the source image data based on a difference between the first inclination angle and the second inclination angle.

3. The imaging system according to claim 1, wherein the controller is further configured to:
detect one or more features in the source image data indicating a rotation of the scope relative to a camera body, wherein the selection of the first subset is responsive to the rotation of the scope.

4. The imaging system according to any one of claims 3, wherein the controller is further configured to:
dewarp the first subset of the source image data generating normalized image data; and
warp or modify the normalized image data in the first subset generating the modified source image data simulating a second inclination angle.

5. The imaging system according to claim 4, wherein the dewarping of the first subset is corrected for one or more distortions or magnifications of the native perspective at the inclination angle.

6. The imaging system according to claim 4, wherein the warping of the normalized image data is distorted, simulating a magnification or distortion at the second inclination angle.

7. The imaging system according to claim 1, wherein the controller is further configured to:
generate a simulated mask enclosed about a perimeter of the first image, wherein the simulated mask includes an orientation cue identifying a direction of the rotation relative to the first image data demonstrating the first portion of the field of view simulating a second inclination angle.

8. The imaging system according to claim 1, wherein the controller is configured to generate the modified source image data by generating a virtual mask superimposed over a field stop mask of the scope about the field of view.

9. The imaging system according to claim 1, wherein the controller is further configured to:
selectively generate second image data demonstrating a second subset of the source image data including a second portion of the field of view simulating a third inclination angle.

10. The imaging system according to claim 1, wherein the first inclination angle is approximately 45° offset from a scope axis of the scope.

11. The imaging system according to claim 10, wherein the controller is further configured to:
selectively output the source data, the first image data, and the second image data to the display screen.

12. The imaging system according to claim 11, wherein the second inclination angle is 30° and the third inclination angle is 70°.

13. The imaging system according to claim 1, wherein the one or more features comprise a field stop mask of the scope demonstrated in the source image data.

14. The imaging system according to claim 1, wherein the generation of the first image data comprises applying an image correction algorithm, wherein the controller is further configured to:
limit a range of the image correction algorithm to the first subset of the source image data for the first image data.

15. The imaging system according to claim 14, wherein the image correction algorithm comprises at least one of an auto-exposure algorithm and a high dynamic range algorithm.

16. A method for controlling a surgical imaging comprising:
controlling the capture of source image data in a field of view at a native perspective at the first inclination angle of a scope at a first inclination angle;
selecting a first subset of the source image including a first portion of the field of view simulating a second inclination angle; and
selectively displaying modified source image data simulating the second inclination angle.

17. The method according to claim 16, wherein the first subset is offset from a focal center of the field of view of the source image data based on a difference between the first inclination angle and the second inclination angle.

18. The method according to claim 16, further comprising:
detecting one or more features in the source image data indicating a rotation of the scope relative to a camera body, wherein the selection of the first subset is responsive to the rotation of the scope.

19. The method according to any one of claims 18, wherein the controller is further configured to:
dewarping the first subset of the source image data generating normalized image data; and
warping or modifying the normalized image data in the first subset generating the modified source image data simulating a second inclination angle.

20. A surgical imaging system comprising:
a scope comprising an optic element aligned at an inclination angle defining a native perspective;
an image sensor configured to capture source image data in a field of view transmitted through the optic element; and
a controller configured to:
control the capture of source image data at the native perspective at the inclination angle;
selectively generate modified image data from the source image data having a simulated view relative to the field of view at the native perspective; and
output the modified image data to a display screen, wherein the simulated view is modified to represent a simulated perspective different than the native perspective, and wherein the source image data comprises a set of pixels and the simulated perspective is generated by remapping light rays aligned with the set of pixels in the source image with an offset center ray aligned with the simulated perspective angle.
